(19) United States
(12) Patent Application Publication
(10) Pub. No.: US 2019/0128788 A1
(43) Pub. Date: May 2, 2019

(54) FLUID SENSOR ASSEMBLY

(71) Applicant: Eaton Intelligent Power Limited, Dublin 4 (IE)

(72) Inventors: Joseph Gerardi, Dryden, MI (US); Richard Ingram, Aurora, NY (US); Michael Sledziona, Ithaca, NY (US)

(21) Appl. No.: 16/306,737

(22) PCT Filed: Jun. 8, 2017
(86) PCT No.: PCT/US2017/036564

§ 371 Date: Dec. 3, 2018

Related U.S. Application Data
(60) Provisional application No. 62/347,397, filed on Jun. 8, 2016.

(51) Int. Cl. G01N 15/02; G01N 33/28

(52) U.S. Cl. CPC G01N 15/0266; G01N 2015/0053; G01N 33/2858

(57) ABSTRACT

A fluid sensor assembly includes a body including a fluid passage, a first sensor connected to the body and directed toward the fluid passage, and a second sensor connected to the body and directed toward the fluid passage. At least one of the first sensor and the second sensor may be configured to transmit a signal into the fluid passage. At least one of the first sensor and the second sensor may be configured to receive at least a deflected version of the signal. The signal may include an ultrasonic pulse. The first sensor may include a focused transmitting transducer and the second sensor may include a non-focused receiving transducer. The fluid passage may include a longitudinal axis and the first sensor may be disposed at an oblique angle relative to the longitudinal axis.

### CROSS-REFERENCE TO RELATED APPLICATION

This application claims the benefit of U.S. Provisional Patent Application Ser. No. 62/347,397, filed on Jun. 8, 2016, the disclosure of which is hereby incorporated herein by reference in its entirety.

### TECHNICAL FIELD

The present disclosure generally relates to fluid systems, fluid sensors, and methods of monitoring fluid systems, such as lubrication systems associated with aircraft.

### BACKGROUND

This background description is set forth below for the purpose of providing context only. Therefore, any aspects of this background description, to the extent that it does not otherwise qualify as prior art, is neither expressly nor impliedly admitted as prior art against the instant disclosure.

Machinery may utilize lubrication systems that can be monitored in real-time for indication of mechanical and fluid degradation. As components of machinery wear, debris particles may break off and enter the lubrication system. Among other things, it may be desirable to determine the particle size distribution and material properties of any contamination in the fluid (e.g., the presence of debris particles). Some monitoring system designs may not be capable of detecting non-metallic wear debris particles. Larger debris particles may indicate more serious potential failure conditions, so a sampling strategy may not be appropriate for larger particles.

An example of an acoustic monitoring system is disclosed in British Patent 1,012,010 (1963). U.S. Pat. Nos. 4,381,674, 4,527,420, and 4,339,944 describe methods of ultrasonic particle sensing. Ultrasonic particle sensing is also described in “Evaluation of an On-Line Ultrasonic Particle Sensor Using Bearing Test Data”, Nemarich, C. P., J. C. Tuner, and Whitesel, H. K., 41st Meeting of the Mechanical Failures Prevention Group, Patuxent River, Md. (1986). U.S. Pat. No. 6,205,848 describes a method and equipment for characterizing particles via acoustic signals. Utilizing ultrasonic transducers for wear debris measurements is described in (1) “Detection of precursor wear debris in lubrication systems”, Edmonds, J., M. Resner, and K. Skharlet, IEEE, 2000; (2) “Helicopter/Tiltrotor Gearbox Debris Monitoring”, Edmonds, J., J. Gerardi, G. Hickman, Navy SBIR Phase I IDI Final Report, 1995).

One or more of the above methods may be limited by the shape of the acoustic beam, which can lead to only a partial volume of the fluid that passes by the transducer being monitored. Particles outside the focus region, including larger size particles indicative of impending failure, might not be detected. Also, one or more of the above methods may not sample fast enough to detect all debris if the flow rate or debris concentration is relatively high. Moreover, some designs may be unable to reliably differentiate between metallic debris and non-metallic debris, air bubbles, or water.

In some designs, a transducer may send an interrogation pulse to a target zone and if a particle is encountered, it may deflect a signal back to the same transducer. Additionally, a transmit crystal ring-down may interfere with returns or echoes received back from targets that are very close to the transducer (e.g., an electrical signal from the transmit pulse may impinge on the time trace of the echo signal and this effect may increases with temperature). Further, with some designs, only small-angle signal deflection departures from the transducer axis may be received be the single transducer.

There is a desire for solutions/options that minimize or eliminate one or more shortcomings of fluid and monitoring systems. The foregoing discussion is intended only to illustrate examples of the present field and should not be taken as a disavowal of scope.

### SUMMARY

In embodiments, a fluid sensor assembly may include a body including a fluid passage, a first sensor connected to the body and directed toward the fluid passage, and/or a second sensor connected to the body and directed toward the fluid passage. At least one of the first sensor and the second sensor may be configured to transmit a signal into the fluid passage. At least one of the first sensor and the second sensor may be configured to receive at least a deflected version of the signal. The signal may include an ultrasonic pulse. The first sensor may include a focused transmitting transducer and the second sensor may include a non-focused receiving transducer. The fluid passage may include a longitudinal axis and the first sensor may be disposed at an oblique angle relative to the longitudinal axis. The oblique angle may be between about 40 degrees and 50 degrees and/or between about 44 and about 46 degrees.

With embodiments, a coil may be disposed at least partially around the fluid passage and may include a longitudinal axis parallel to a longitudinal axis of the fluid passage. A first sensor may be disposed at a first oblique angle relative to a longitudinal axis of the fluid passage and may be disposed at a second oblique angle relative to a transverse axis of the body. A second sensor may be aligned with the transverse axis of the body. The first sensor may include a focused transmitting transducer. The first sensor may include a first crystal and the second sensor may include a second crystal.

In embodiments, a fluid sensor assembly may include a third sensor and a fourth sensor. The third sensor may be configured to transmit a second signal into the fluid passage and the fourth sensor may be configured to receive at least one of a deflected version of the second signal and the deflected version of the signal. A fluid sensor assembly may include a third sensor configured to receive the deflected version of the signal. A third sensor may be substantially coaxial with the second sensor. In embodiments, a fluid sensor assembly may include a fluid conduit connected to an inlet of the body. The fluid conduit may include a vertical portion, a bent portion, and/or an outlet. The outlet may be disposed at an oblique angle relative to a longitudinal axis of the fluid passage. A first sensor and a second sensor may be disposed in a V-shaped configuration.

With embodiments, a method of sensing particles in a fluid may include providing a fluid sensor assembly. The fluid sensor assembly may include a housing including a fluid passage, a first sensor connected to the housing and directed toward the fluid passage, and/or a second sensor connected to the housing and directed toward the fluid passage. A method of sensing particles may include transmitting, via the first sensor, a signal into the fluid passage, receiving, via the second sensor, a deflected version of the signal that has deflected or scattered after encountering a debris particle, and/or detecting the debris particle according the deflected version of the signal. At least one of the first sensor and the second sensor may be disposed at a first oblique angle relative to a longitudinal axis of the fluid passage and/or at a second oblique angle relative to a transverse axis of the housing. A method of sensing particles may include determining at least one of a size, a shape, and a material of the debris particle according to the deflected version of the signal. A method of sensing particles may include sensing the debris particle via an inductive sensor connected to the housing. A method of sensing particles may include transmitting, via the second sensor, a second signal into the fluid passage and/or receiving, via the first sensor, a deflected version of the second signal that has deflected or scatter after encountering a second debris particle. A method of sensing particles may include providing an electronic control unit (ECU), providing an inductive sensor, and/or applying, via the ECU, signal processing methods to information from the first sensor, information from the second sensor, and/or information from the inductive sensor to determine a size of the debris particle and/or whether the debris particle includes metal. A method of sensing particles may include transmitting, via the first sensor, a second signal into the fluid passage, and receiving, via the second sensor, a deflected version of the second signal that has deflected or scattered after encountering the debris particle.

Various aspects of the present disclosure will become apparent to those skilled in the art from the following detailed description of the various embodiments, when read in light of the accompanying drawings.

### BRIEF DESCRIPTION OF THE DRAWINGS

FIG. 1 is a perspective view generally illustrating an embodiment of a fluid sensor assembly according to teachings of the present disclosure.

FIGS. 2, 3 and 4 are cross-sectional views generally illustrating embodiments of fluid sensor assemblies according to teachings of the present disclosure.

FIG. 5 is a perspective view generally illustrating an embodiment of a fluid sensor assembly according to teachings of the present disclosure.

FIGS. 6 and 7 are cross-sectional views generally illustrating bodies of embodiments of fluid sensor assemblies according to teachings of the present disclosure.

FIG. 8 is a side view generally illustrating an embodiment of a body of a fluid sensor assembly according to teachings of the present disclosure.

FIG. 9 is a side view generally illustrating an embodiment of a sensor according to teachings of the present disclosure.

FIG. 10 is a cross-section view generally illustrating an embodiment of a cartridge sensor according to teachings of the present disclosure.

FIGS. 11 and 12 are cross-sectional views generally illustrating bodies of embodiments of fluid sensor assemblies according to teachings of the present disclosure.

FIG. 13 is a side view generally illustrating embodiments of sensors according to teachings of the present disclosure.

FIG. 14 is a side view generally illustrating an embodiment of a fluid conduit according to teachings of the present disclosure.

FIG. 15 is a cross-sectional view generally illustrating an embodiment of a fluid conduit according to teachings of the present disclosure.

### DETAILED DESCRIPTION

Reference will now be made in detail to embodiments of the present disclosure, examples of which are described herein and illustrated in the accompanying drawings. While the present disclosure will be described in conjunction with embodiments and/or examples, it will be understood that they are not intended to limit the present disclosure to these embodiments and/or examples. On the contrary, the present disclosure is intended to cover alternatives, modifications and equivalents, which may be included within the spirit and scope of the present disclosure as defined by the appended claims.

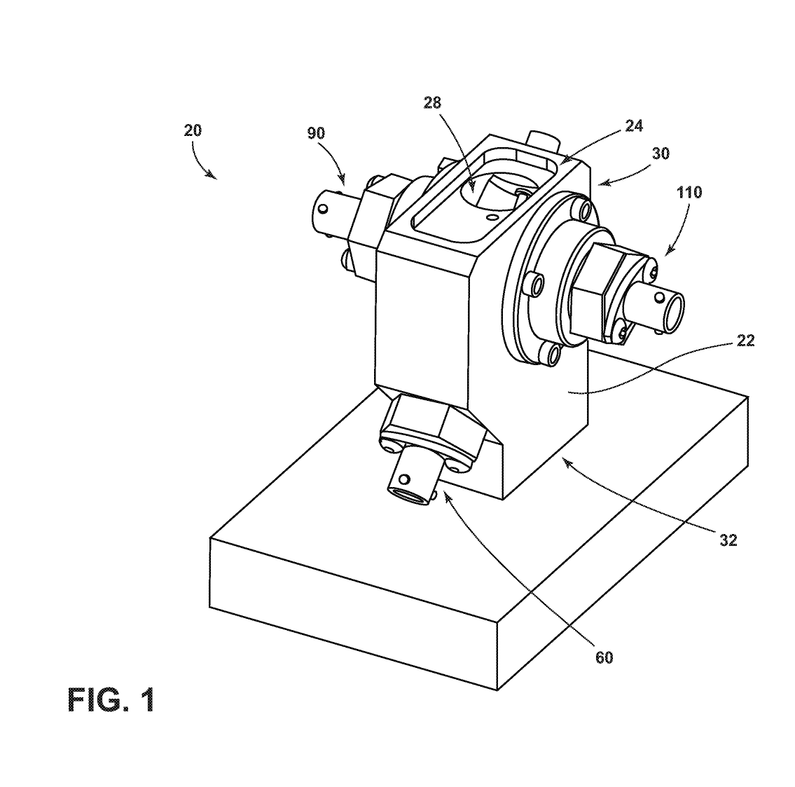

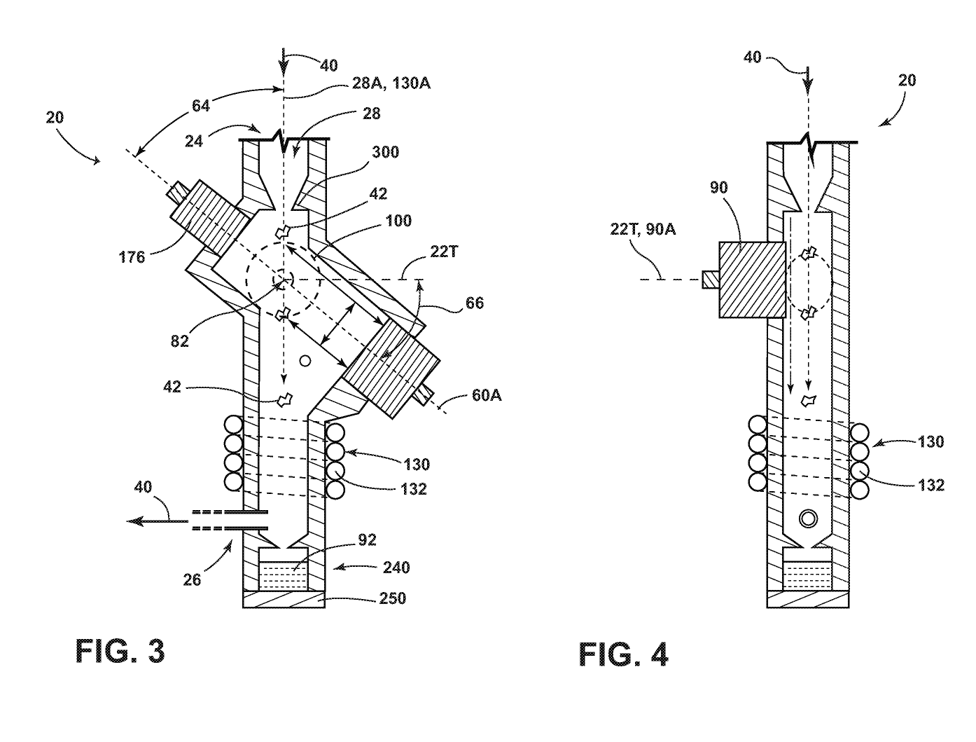

In embodiments, such as generally illustrated in FIGS. 1, 2, 3, 4, and 5, a fluid sensor assembly 20 may include a body/housing 22 and a plurality of sensors, such as a first sensor 60, a second sensor 90, and/or a third sensor 110. Body 22 may include an inlet 24, an outlet 26, and/or a fluid passage 28 providing fluid communication between inlet 24 and outlet 26. Inlet 24 may be disposed at or about a first end 30 of body 22 (e.g., a top) and/or outlet 26 may be disposed at or about a second end 32 of body 22 (e.g., a bottom). Fluid passage 28 may include a longitudinal axis 28A that may, for example, be aligned and/or parallel with a vertical direction. Body 22 may, for example, be configured to hold or store, at least temporarily a volume of fluid 40. The fluid may contain particles 42. In embodiments, fluid sensor assembly 20 may be utilized in connection with a fluid separator, such as the fluid separator described in commonly owned U.S. Pat. No. 7,288,139, which is hereby incorporated by reference herein in its entirety. For example and without limitation, debris particles 42 may be separated or at least directed into a particle receiving area and fluid sensor assembly 20 may be connected to the particle receiving area. Fluid 40 in body 22 may be relatively still (e.g. not flowing) during sensing, and particles 42 may fall within body 22 and bubbles may rise (e.g., due to gravity/density differences).

With embodiments, outlet 26 may be configured as a fluid exchange/renewal port that may allow for periodic exchange of fluid 40 within body 22, which may provide a more accurate and/or up-to-date sample of fluid 40 in a fluid system. Sensing may be suspended during fluid exchange. A fluid exchange may include a substantially complete purging of fluid 40 out of body 22 and new fluid 40 entering body (e.g., via inlet 24). A fluid exchange may involve some degree of backflow from outlet 26 to inlet 24 and may be gentle enough not to push larger particles 42 back through inlet and into the fluid system (e.g., back into a separator).

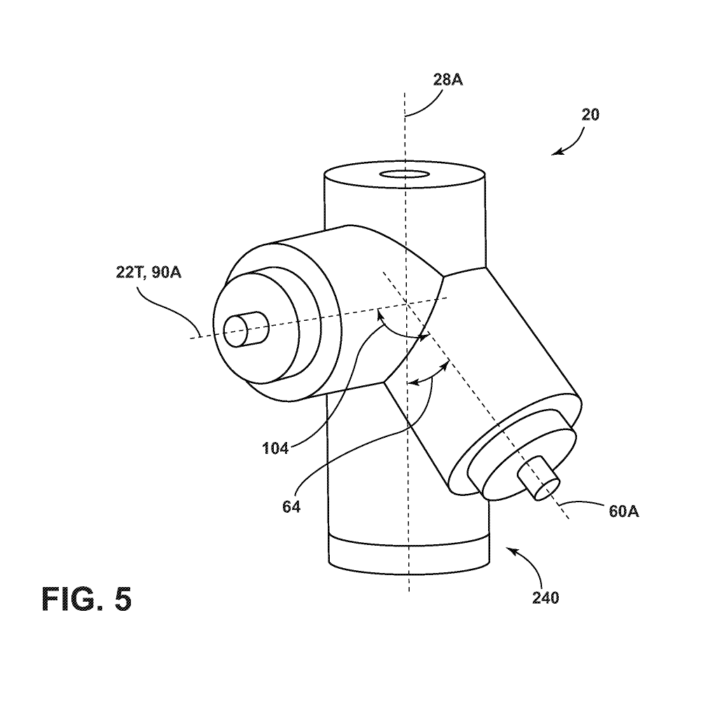

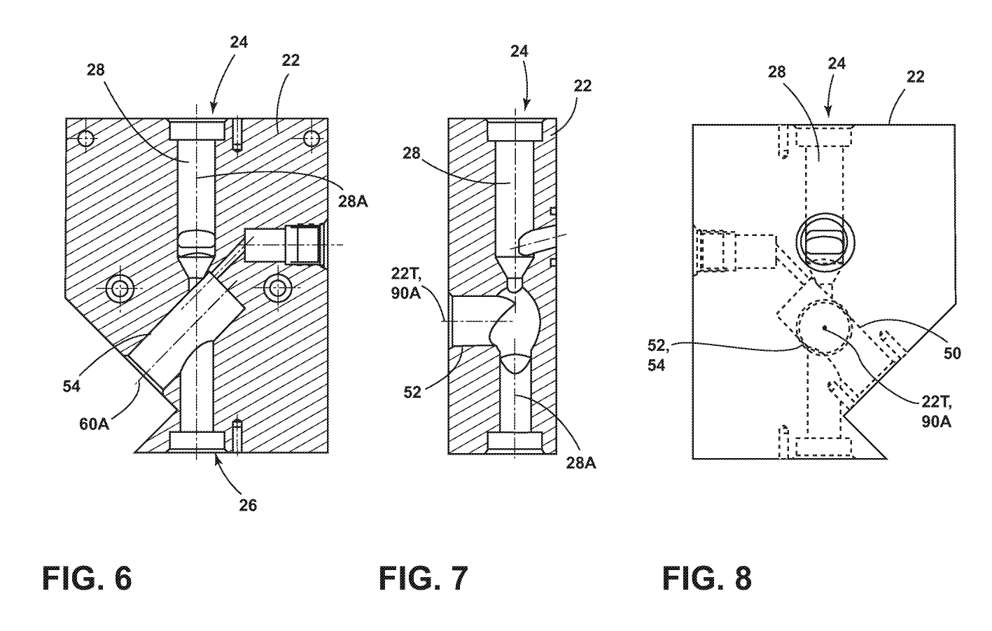

With embodiments, such as generally illustrated in FIGS. 1-8, body 22 may include a first recess or aperture 50 configured to at least partially receive first sensor 60 and/or may include a second recess or aperture 52 configured to at least partially receive second sensor 90.

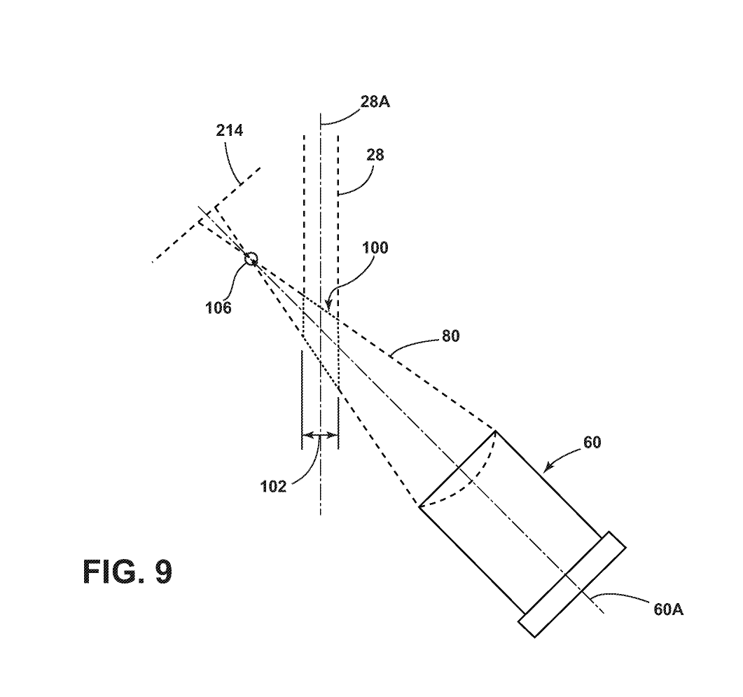

With embodiments, such as generally illustrated in FIGS. 1-5, a sensor may include one or more of a variety of shapes, sizes, configurations, and/or materials. For example and without limitation, first sensor 60 may be configured to transmit a signal or signals 62 and/or second sensor 90 may be configured to receive a deflected or echoed version 92 of signal or signals 62. In embodiments, first sensor 60 may include a transmitting transducer configured to transmit (e.g., generate, propagate, emit, etc.) an acoustic signal/wave 62 toward and/or into fluid passage 28. For example and without limitation, first sensor 60 may include a piezoelectric crystal that may be pulsed at a frequency from about 2.5 MHz to about 5 MHz. The crystal may, for example, include a frequency of about 2.5 MHz or of about 5 MHz. First sensor 60 may be configured as a focused transducer, include a focused acoustic lens 72, and/or provide a focus zone 80 (see, e.g., FIG. 9). First sensor 60 and second sensor 90 may be disposed in a bistatic configuration. First sensor 60 may be connected with and/or at least partially disposed in first aperture 50 such that a first sensor axis 60A is disposed at angle 64 relative to longitudinal axis 28A. For example and without limitation, angle 64 may be an oblique angle relative to longitudinal axis 28A, such as an angle of about 40 degrees to 50 degrees, about 44 degrees to about 46 degrees, and/or about 45 degrees. A transverse axis 22T of body 22 may be perpendicular to longitudinal axis 28A. First sensor axis 60A may be aligned at an angle 66 (e.g., an oblique angle) relative to transverse axis 22T, for example and without limitation, an angle of about 40 degrees to 50 degrees, about 44 degrees to about 46 degrees, and/or about 45 degrees. First sensor axis 60A may intersect with longitudinal axis 28A at an intersection point 82 that may be disposed in fluid passage 28 (e.g., centered in passage 28). First sensor 60 may be directed generally toward first end 30 of body 22 (e.g., a first/transmit end 68 of first sensor 60 may be disposed closer to first end 30 of body 22 than an opposite end 70 of first sensor 60).

In embodiments, second sensor 90 may include a receiving transducer configured to receive acoustic signal/wave 62, and/or a version or portion thereof, transmitted by first sensor 60. For example and without limitation, first sensor 60 may transmit signal 62 in a first direction toward a target area or portion 100 of fluid passage 28. Target area 100 may be centered on longitudinal axis 28A and/or intersection point 82. If a particle 42 is present in the target portion 100, signal 62 may deflect off of the particle 42 and signal 62 may then be directed in one or more different directions, at least one direction of which may be toward second sensor 90. Second sensor 90 may be configured to receive a deflected version or portion 92 of signal 62 that has encountered a particle 42. Second sensor 90 may be configured to convert or translate a version or portion 92 of signal 62 received by second sensor 90 (e.g., an acoustic signal/wave) into an electrical signal that may correspond to one or more properties of the particle 42 (e.g., size, shape, etc.). With embodiments, second sensor 90 may be configured as a non-focused receiving transducer. In embodiments, second sensor 90 may be disposed as close to target area 100 as possible (e.g., at or just outside a drop diameter 102).

With embodiments, second sensor 90 may be connected to and/or at least partially disposed in second aperture 52 such that a second sensor axis 90A may be disposed in parallel with and/or may be coaxial with transverse axis 22T. Second sensor axis 90A may intersect with longitudinal axis 28A and/or first sensor axis 60A. For example and without limitation, longitudinal axis 28A, first sensor axis 60A, and second sensor axis 90A may pass through relatively small common area and/or may intersect at a common point (e.g., intersection point 82). Intersection point 82 may be disposed in target area or portion 100, such as at or about the center of target area or portion 100. First sensor 60 and second sensor 90 may be disposed at one or more angles relative to each other. For example and without limitation, first sensor 60 and second sensor 90 may be disposed at an angle 104 of about 90 degrees relative to each other (e.g., circumferentially/viewed along longitudinal axis 28A). In embodiments, a plurality of signals 62 may be transmitted and encounter (and deflect from) the same particle 42, which may provide additional information about the particle 42. First sensor and second sensor may be discrete, separate, and/or independent from each other.

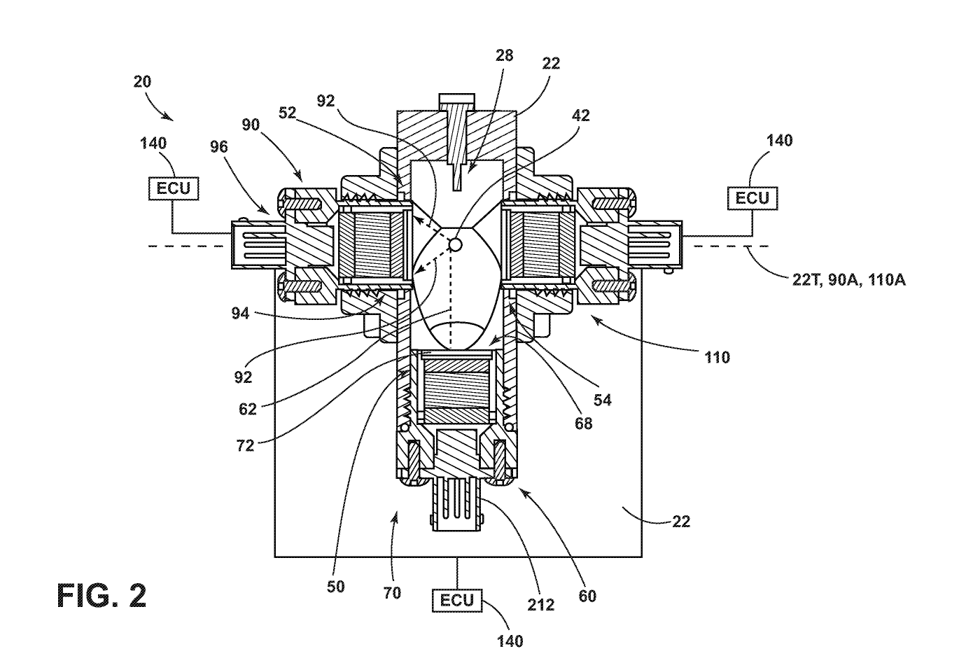

In embodiments, fluid sensor assembly 20 may include a third sensor 110 (see, e.g., FIGS. 1 and 2). Body 22 may include a third recess 54 that may be configured for connection with and/or to at least partially receive third sensor 110. Third sensor 110 may be connected with and/or at least partially disposed in third recess 54 such that a third sensor axis 110A may be disposed substantially in parallel with transverse axis 22T, in parallel with second sensor axis 90A, and/or coaxial with second sensor axis 90A (e.g., second sensor 90 and third sensor 110 may be coaxial). Third sensor 110 may include a receiving transducer that may be configured to receive acoustic signals/waves 62. For example and without limitation, third sensor 110 may be configured to receive a deflected version or portion 92 of signal/wave 62 that has encounter/deflected off of a particle 42. A deflected version or portion 92 of signal/wave 62 received by third sensor 110 may have deflected in a different direction from particle 42 than a deflected version or portion 92 of signal/wave 62 received by second sensor 90. Third sensor 110 may be utilized in addition to and/or instead of second sensor 90. Utilizing third sensor 110 with second sensor 90 may allow for a greater portion of signal/wave 62 to be received after deflection, which may provide more or better information about a particle 42, especially for particles 42 of non-uniform shapes (e.g., non-spherical particles). For example and without limitation, if a particle 42 includes a facet and a deflected version 92 of signal 62 deflects to a relatively minimal extent or does not deflect toward second sensor 90, some of the deflected version 92 may be deflected to and/or received by third sensor 110 (or vice versa). With embodiments, third sensor 110 may be connected in parallel with second sensor 90.

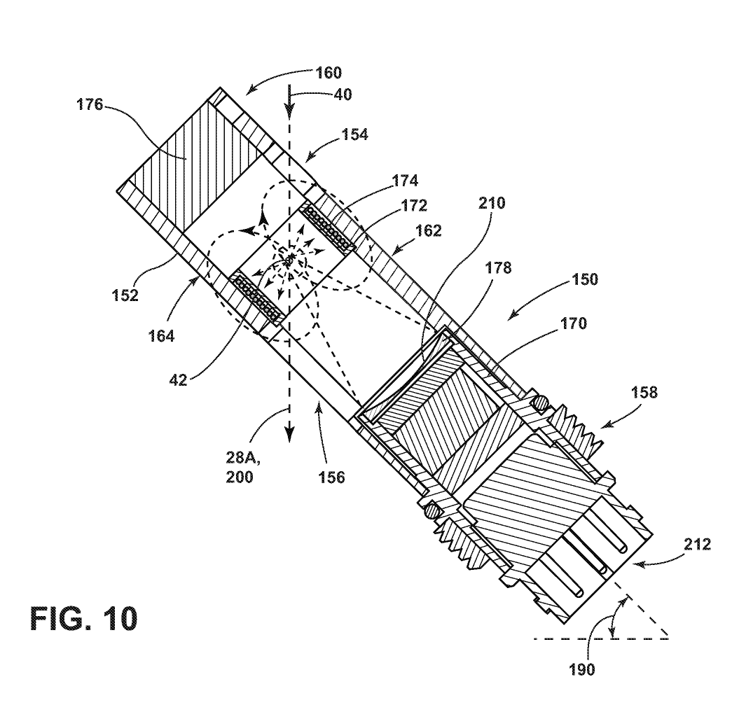

In embodiments, such as generally illustrated in FIGS. 3, 4, 10, and 12, a fluid sensor assembly 20 may include a fourth sensor 130. Fourth sensor 130 may include an inductive sensor that may be configured to sense metallic particles 42 in body 22 and/or fluid passage 28. For example and without limitation, fourth sensor 130 may include a coil 132 that may be disposed around at least a portion of body 22 such that a fourth sensor axis 130A may be substantially aligned with (e.g., coaxial with) longitudinal axis 28A of fluid passage 28 (see, e.g., FIGS. 3, 4, and 12). Additionally or alternatively, a coil 132 may be disposed within body 22 and/or fluid passage 28 such that fourth sensor axis 130A is substantially aligned with longitudinal axis 28A of fluid passage 28 (see, e.g., FIG. 10). An electrical current may be provided to coil 132 and if a metal particle 42 moves through coil 132 (e.g., radially within coil 132), fourth sensor 130 may detect a change in inductance that may correspond to a size, shape, and/or material of the particle 42. Fourth sensor 130 may not be configured for detecting non-metal particles 42.

With embodiments, such as generally illustrated in FIG. 2, an electronic control unit (ECU) 140 may be connected with one or more sensors (e.g., first sensor 60, second sensor 90, third sensor 110, fourth sensor 130, a cartridge sensor 150, a fifth sensor 230, a sixth sensor 270, and/or other sensors). ECU 140 may be configured to receive information (e.g., data, signals, etc.) from one or more sensors and ECU 140 may determine one or more of (i) whether particles 42 are present in fluid passage, (ii) a size associated with any such particle 42, (iii) a shape of any such particle 42, and/or (iv) a material of any such particle 42 (e.g., metal or non-metal). For example and without limitation, if a particle 42 is detected via first sensor 60 and second sensor 90 and is also detected by fourth sensor 130, ECU 140 may determine the particle 42 is metal. If a particle 42 is detected via first sensor 60 and second sensor 90, but is not detected by fourth sensor 130, ECU 140 may determine that the particle 42 is non-metal. ECU 140 may be configured to control operation of one or more sensors (e.g., first sensor 60, second sensor 90, third sensor 110, fourth sensor 130, cartridge sensor 150, fifth sensor 230, sixth sensor 270, and/or other sensors). ECU 140 may be configured to apply one or more signal processing methods to received information (e.g., information received from one or more of sensors 60, 90, 110, 130, 150, 230, 270). Signal processing methods may include various functions, for example and without limitation, averaging, fuzzy logic, and/or pattern recognition.

In embodiments, ECU 140 may include an electronic controller and/or include an electronic processor, such as a programmable microprocessor and/or microcontroller. In embodiments, ECU 140 may include, for example, an application specific integrated circuit (ASIC). ECU 140 may include a central processing unit (CPU), a memory, and/or an input/output (I/O) interface. ECU 140 may be configured to perform various functions, including those described in greater detail herein, with appropriate programming instructions and/or code embodied in software, hardware, and/or other medium. In embodiments, ECU 140 may include a plurality of controllers. In embodiments, ECU 140 may be connected to a display.

With embodiments, such as generally illustrated in FIG. 10, a sensor 150 may include a cartridge configuration. A cartridge sensor 150 may include a body 152, a transmit transducer 170, a receiver 172, an inductive coil 174, a dirtiness sensor 176, and/or an acoustic lens 178. Body 152 may include a generally cylindrical or tubular shape that may be connected to sensor assembly body 22 at an angle, such as angle 190 (e.g., an oblique angle). Body 152 may include an inlet 154, an outlet 156, a first end 158, a second end 160, a first side 162 (e.g., a top), and/or a second side 164 (e.g., a bottom). Inlet 154 may include a slot or aperture in first side 162 and/or outlet 156 may include a slot or aperture in second side 164. Bubbles may purge via inlet 154. Inlet 154 and outlet 156 may be axially spaced from each other such that if body 152 is disposed at angle 190 (e.g., upon connection with body 22), a generally vertical fluid path 200 (e.g., a drop path) may be provided through body 152 from inlet 154 to outlet 156. Fluid path 28 of body 22 of fluid assembly 20 may at least partially align with and/or include fluid path 200 of body 152 of cartridge sensor 150.

In embodiments, transmitting transducer 170 may be disposed at or about first end 158 of body 152 and/or at least partially within body 152. Transmitting transducer 170 may be configured to transmit an acoustic signal/wave 62 into body toward second end 160 and/or may provide a focused acoustic field. Acoustic lens 178 may be disposed with body 152 at or about a transmitting end 158 of transmitting transducer 170 between transmitting transducer 170 and second end 160 of body 152. Particles 42 that move away from longitudinal axis 28A may fall onto transmitting transducer 170 and may slide down a tapered face 210 of transmitting transducer 170 and/or of acoustic lens 178 until the particles 42 reach outlet 156.

In embodiments, receiver 172 may include a generally ring-like or cylindrical configuration and may include, for example, a piezoelectric ring receiver (e.g., lead zirconate titanate or PZT) that may be configured to receive signals (e.g., deflected signals 92) from a plurality of directions. Receiver 172 may be disposed at least partially between inlet 154 and outlet 156 (e.g., axially) such that fluid path 200 may extend from inlet 154 through receiver 172 to outlet 156. For example and without limitation, fluid 40 and/or particles 42 may, at least initially, flow/fall into inlet 154, through receiver 172, and to outlet 156. Inductive coil 174 may be disposed at least partially between inlet 154 and outlet 156 (e.g., axially) such that fluid path 200 may extend from inlet 154 through inductive coil 174 to outlet 156. For example and without limitation, metal particles 42 that flow/fall into inlet 154 may fall through inductive coil 174, which may sense such metal particles 42, and metal particles 42 may continue to flow/fall to outlet 156. Inductive coil 174 may be coaxial with receiver 172 and/or inductive coil 174 and receiver 172 may be integrated and/or connected with each other. Dirtiness sensor 176 may be connected and/or disposed at or about second end 160 and may be directed toward first end 158, transmit transducer 170, and/or acoustic lens 178.

With embodiments, if fluid 40 is present in body 152 (e.g., if fluid 40 flows into body 152 through inlet 154), transmitting transducer 170 may transmit a signal 62 into body 152. If a particle 42 is present in body 152 and/or fluid 40, signal 62 may deflect from/off of the particle 42. Receiver 172 may receive at least a portion of the deflected signal 92 and may generate an electrical signal that may correspond to one or more characteristics of the particle 42 (e.g., size, shape, material, etc.). Receiver 172 may be connected to a connector 212 that may be connected at or about first end 158 of body 152 and/or may be connected to transmitting transducer 170. Connector 212 may, for example, be connected to ECU 140. If a metal particle 42 is present in or near inductive coil 174, an inductance of inductive coil 174 may change according to one or more characteristics of the particle 42 (e.g., size, shape). Additionally or alternatively, inductive coil 174 may be connected to connector 212 and/or ECU 140. With embodiments, cartridge sensor 150 may be connected with body 22 of fluid sensor assembly 20 and may be used in conjunction with and/or instead of first sensor 60, second sensor 90, and/or fourth sensor 130.

With embodiments, cartridge body 152 may include a generally square-shaped cross section. In such embodiments, receiver 172 and/or inductive coil 174 may include corresponding square-shaped cross sections.

In embodiments, a reverse configuration may include first sensor 60 including a receiving transducer and/or second sensor 90 including a transmitting transducer. For example and without limitation, second sensor 90 may transmit, without a focal zone, a signal 62 that may deflect from and/or off of a particle 42 and a deflected version or portion 92 of the signal 62 may be received by first sensor 60. In a reverse configuration, a transmitting transducer (e.g., second sensor 90) may be disposed closer to a target zone 100 (e.g., relative to first sensor 60 in a forward configuration), which may increase the energy of the transmit signal 62 that reaches a particle and/or the signal(s) 92 deflected/scattered from a particle 42. Additionally or alternatively, some of the acoustic energy that may have missed the receiving transducer (e.g., second sensor 90 in the forward configuration) may be at least indirectly received by first sensor 60 since signals 92 may deflect or scatter from a mounting pocket wall 214 opposite first sensor 60. A reverse configuration may be utilized, for example, to sense relatively small particles 42 (e.g., about 50μ to 100μ compared to 100 μin to 1000 μin). Sensing smaller particles 42 may be conducted via sampling as a full count of smaller particles may not be desired.

With embodiments, fluid sensor assembly 20 may be operated in both a bistatic sensor mode (e.g., for large particles) and a monostatic mode (e.g., for smaller particles, with first sensor 60 and/or second sensor 90 functioning as both a transmitting transducer and a receiving transducer). For example and without limitation, in one sensing cycle, a bistatic sensor mode may be used and then a monostatic mode may be used, and such a cycle may repeat. In a bistatic mode, ECU 140 may be configured to register multiple particles/hits on the same particle 42 such movement of a particle 42 can be tracked to distinguish particles from bubbles. If a large particle 42 is present during the monostatic mode, ECU 140 may identify the large particle based on its rate of descent and may ignore it (until the bistatic mode resumes).

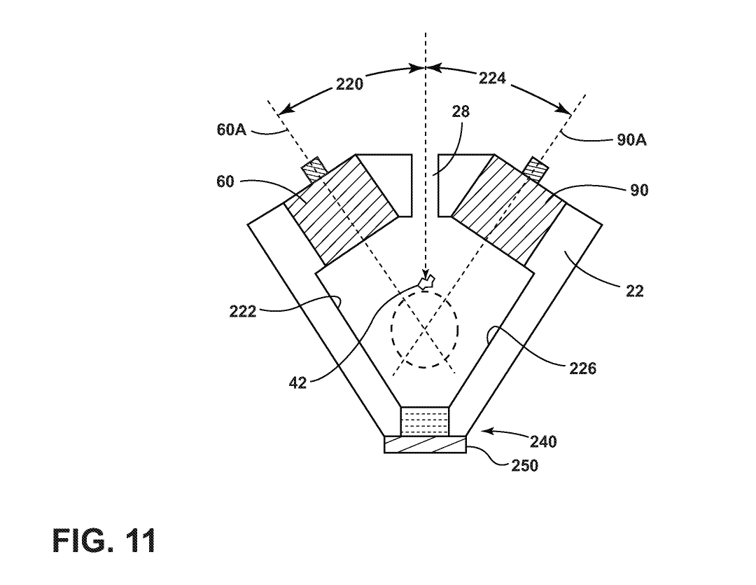

In embodiments, such as generally illustrated in FIGS. 11 and 12, first sensor 60 and second sensor 90 may be disposed in a V-shaped configuration and/or body 22 may include a V-shaped configuration. For example and without limitation, first sensor 60 may be connected to body 22 such that first sensor axis 60A is disposed at a first acute angle 220 relative to longitudinal axis 28A, which may include first sensor 60 pointing generally downward with a transmitting end 68 of first sensor 60 closer to longitudinal axis 28A than an opposite end 70 of first sensor 60. A first inner wall 222 of body 22 may extend at first acute angle 220 and/or may be disposed opposite second sensor 90. Second sensor 90 may be connected to body 22 such that second sensor axis 90A may disposed at a second acute angle 224 relative to longitudinal axis 28A, which may include second sensor 90 pointing generally downward with a receiving end 94 of second sensor 90 closer to longitudinal axis 28A than an opposite end 96 of second sensor 90. A second inner wall 226 of body 22 may extend at second acute angle 224 and/or may be disposed opposite first sensor 60. First sensor 60 and second sensor 90 may be disposed at opposite sides of longitudinal axis 28A and may be substantially aligned in a common plane (e.g., a vertical plane). First acute angle 220 and second acute angle 224 may be substantially the same. For example and without limitation, first acute angle 220 and second acute angle 224 may be about 30 degrees to 40 degrees, about 33 degree to about 37 degrees, and/or about 35 degrees. An angle between first sensor axis 60A and second sensor axis 90A (e.g., a sum of angle and angle) may, for example, be about 70 degrees. In a V-shaped configuration, particles 42 may not accumulate at or near first sensor 60 and/or second sensor 90 (e.g., gravity may pull particles 42 generally downward/away from first sensor 60 and/or second sensor 90). A V-shaped configuration may provide a longer fluid path 28 (e.g., a drop path) within body 22 relative to other configurations, such as the configurations generally illustrated in FIGS. 1-10. With embodiments, first sensor 60 and second sensor 90 may be disposed perpendicularly to fluid path axis 28A. For example and without limitation, first sensor axis 60A and second sensor axis 90A may be disposed at an angle relative to each other and/or be substantially aligned in the common plane (e.g., a horizontal plane).

With embodiments, fluid sensor assembly 20 may include an array of sensors. For example and without limitation, as generally illustrated in FIG. 12, fluid sensor assembly 20 may include first sensor 60, second sensor 90, third sensor 110, fourth sensor 130, and/or a fifth sensor 230. Fifth sensor 230 may be configured in the same or a similar manner as first sensor 60. For example and without limitation, fifth sensor 230 may include a focused transmitting transducer configured to transmit an acoustic signal/wave 62 toward and/or into fluid passage 28. First sensor 60 and second sensor 90 may be disposed in a first V-configuration and third sensor 110 and fifth sensor 230 may be disposed in a second V-shaped configuration that may mirror the first V-configuration. For example and without limitation, first sensor 60, second sensor 90, third sensor 110, and fifth sensor 230 may be disposed in a X-shaped configuration. First sensor 60 and fifth sensor 230 may be substantially aligned with each other (e.g., coaxial) and/or second sensor 90 and third sensor 110 may be substantially aligned with each other. First sensor 60 and third sensor 110 may be disposed at a first side of longitudinal axis 28A and/or second sensor 90 and fifth sensor 230 may be disposed at a second side of longitudinal axis 28A. With embodiments, fifth sensor 230 may be used in other configurations, such as those generally illustrated in FIGS. 1-5. For example and without limitation, fluid sensor assembly 20 may include one or more of first sensor 60, second sensor 90, third sensor 110, fourth sensor 130, and/or a fifth sensor 230.

In embodiments, such as generally illustrated in FIGS. 3, 4, 5, 12, and 15, fluid sensor assembly 20 may include a debris port/pocket 240. Debris port 240 may be configured to receive and/or at least temporarily store particles 42 that flow/fall through fluid path 28. A cover 250 may be connected to debris port 240 and may permit access to debris particles 42 in debris port 240 (e.g., debris particles 42 may be removed for study). Debris port 240 and/or cover 250 may be magnetic.

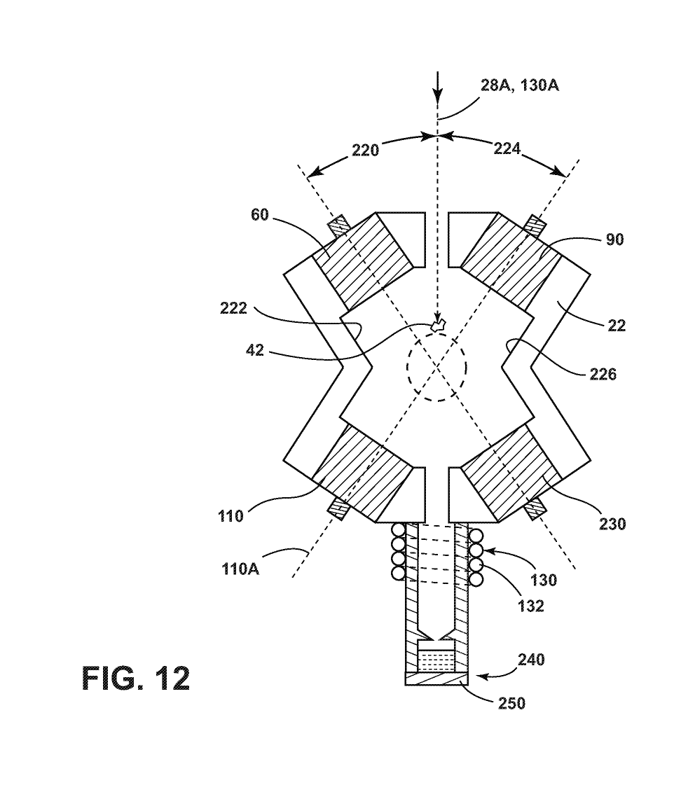

With embodiments, such as generally illustrated in FIG. 13, fluid sensor assembly 20 may include a reflector 260 and a reverse configuration of first sensor and second sensor. Reflector 260 may be disposed opposite first sensor 60 (which may include/be configured as a receiving transducer). Reflector 260 may include a focus with a radius 262 that may extend to first sensor 60. Reflector 260 may be configured to reflect signals (e.g., signal/portion 92′) toward first sensor 60. For example and without limitation, a signal 62 transmitted from second sensor 90 may result in two return signals 92, 92′ reaching first sensor, such as an original, deflected signal 92 and a reflected signal 92′. The reflected signal 92′ may be smaller than deflected signal 92 and/or may reach first sensor 60 after deflected signal 92. A time delay between deflected signal 92 and reflected signal 92′ may be utilized to compensate for electrical noise. Additionally or alternatively, fluid sensor assembly 20 may include a sixth sensor 270 that may be disposed opposite first sensor 60. Sixth sensor 270 may include reflector 260 and/or may include a concave surface that may function as reflector 260. Sixth sensor may include a receiving transducer that may function as a trigger source to activate a listening mode of first sensor 60. The listening mode may correspond to a narrowly defined time window just before the arrival of a retro-reflection event from reflector 260 of sixth sensor 270. ECU 140 may sample a signal received by first sensor 60 to determine a peak in the signal. A field 272 of sixth sensor 270 may be low-noise or noise-free, such that deflected signal 92 and reflected signal 92′ can be utilized and/or averaged, and ECU 140 may determine whether signals 92, 92′ are a result of a bubble (if moving up) or a particle 42 (if falling/moving down).

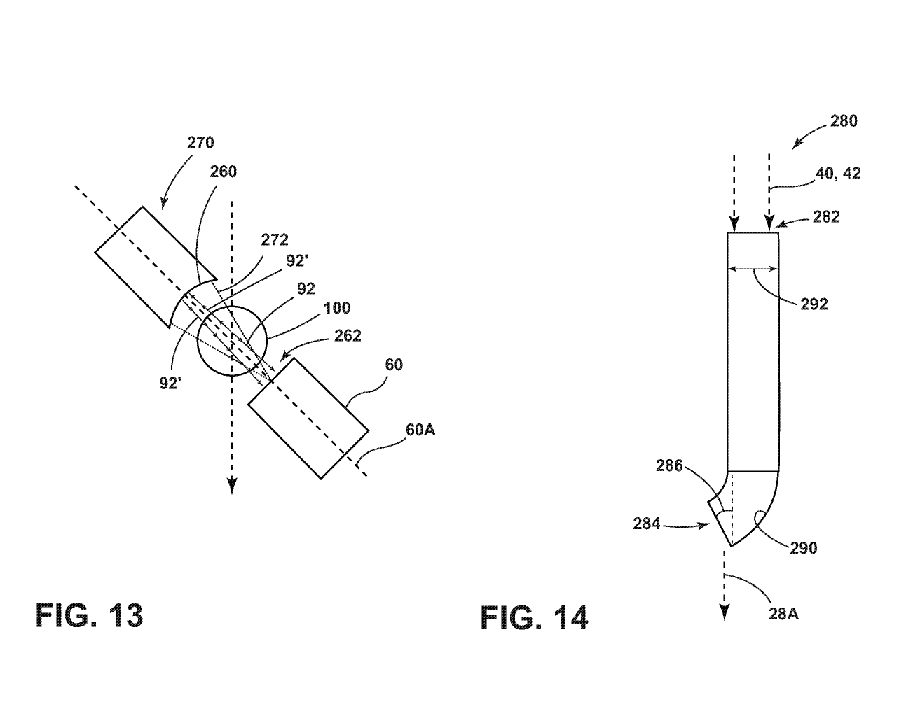

With embodiments, such as generally illustrated in FIG. 14, fluid sensor assembly 20 may include a fluid conduit 280 that may be connected to inlet 24 of body 22 and/or at least partially disposed in body 22. Fluid conduit 280 may include an inlet 282 may open substantially vertically upward and/or and outlet 284 that may open generally downward at an angle 286 relative to a vertical direction. For example and without limitation, fluid conduit 280 may include a bent section 288 that may bend at about 45 degrees and outlet 284 may open at an angle of about 45 degrees. If a particle 42 falls/flows from inlet 282 toward outlet 284, the particle 42 may contact an upward facing inner surface 290 of bent section 288 and may slide down surface 290 until the particle 42 falls out of outlet 284. Outlet 284 may be substantially aligned with longitudinal axis 28A of fluid passage 28 such that particles 42 exiting outlet 284 may fall substantially along longitudinal axis 28A, which may make sensing of particles 42 more consistent. Facilitating particles 42 falling along longitudinal axis 28A may reduce an expected drop zone 102 (e.g., a diameter around longitudinal axis 28A in which particles 42 are expected to fall, such as generally illustrated in FIG. 9), which may permit disposing sensors closer to longitudinal axis 28A (e.g., provide more energy to and/or receive more energy from particles 42) and/or allow for one or more sensors to include a semi-focus instead of a cylindrical field (or other shape). First sensor 60 may include a focal point or full focus zone 106. A fluid conduit 280 may be utilized in addition to and/or as an alternative to a constriction 300 at or about inlet 24 (see, e.g., FIGS. 3 and 4). Fluid conduit 280 may be less prone to clogging (e.g., relative to designs including constrictions) as an inner diameter 292 of fluid conduit 280 may be substantially constant from inlet 282 to outlet 284 (e.g., about 0.2 inches).

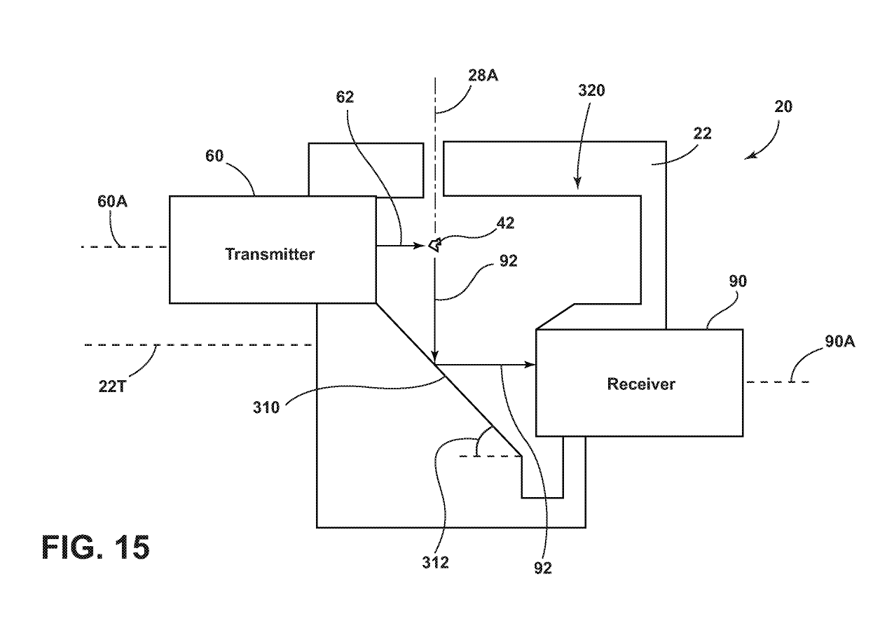

In embodiments, such as generally illustrated in FIG. 15, body of fluid sensor assembly 20 may include a reflection wall 310 that may be aligned with longitudinal axis 28A (e.g., longitudinal axis 28A may intersect with tapered wall 310). First sensor 60 may include a transmitting transducer and may be disposed above reflection wall 310 such that first sensor axis 60A may be substantially parallel with a transverse axis 22T. Second sensor 90 may include a receiving transducer and/or be disposed such that second sensor axis 90A may be substantially parallel with transverse axis 22T and/or may intersect with reflection wall 310 at or about the same point as longitudinal axis 28A. Reflection wall 310 may include, for example, a taper angle 312 of about 45 degrees. In embodiments, reflection wall 310 may include a curvature and/or other shapes. First sensor 60 may transmit a signal 62 that may deflect off of or from a particle 42 and a deflected signal or portion 92 may reflect off of reflection wall 310 toward second sensor 90. Such a configuration may provide similar functionality as if second sensor 90 was mounted vertically upward and aligned with longitudinal axis 28A, but particles 42 may not accumulate on and/or obstruct second sensor 90.

In embodiments, a debris port 240 may be disposed below reflection wall 310. Particles 42 may fall until they reach reflection wall 310 and may slide down reflection wall 310 until the particles 42 fall into debris port 240.

With embodiments, body 22 may include a delay cavity 320 that may be disposed opposite first sensor 60. Signals 62 from first sensor 60 that pass beyond target zone 100 (e.g., significantly beyond longitudinal axis 28A) may enter delay cavity 320 and may become delayed enough and/or may attenuate enough not to contribute to signal noise.

With embodiments, a bistatic configuration of first sensor 60 and second sensor 90 may provide one or more advantages relative to other designs (e.g., monostatic designs). For example and without limitation, a ring-down of the transmit transducer may not be material as the transmit transducer (e.g., first sensor 60) may be acoustically isolated from the receiver (e.g., second sensor 90), which may allow for a larger gain to be used. The receiver (e.g., second sensor 90) may be disposed closer to the target zone 100 as it might be desirable to dispose a transmit transducer at a minimum distance from the target zone 100.

Various embodiments are described herein for various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments.

Reference throughout the specification to “various embodiments,” “with embodiments,” “in embodiments,” or “an embodiment,” or the like, means that a particular feature, structure, or characteristic described in connection with the embodiment is included in at least one embodiment. Thus, appearances of the phrases “in various embodiments,” “with embodiments,” “in embodiments,” or “an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features, structures, or characteristics of one or more other embodiments without limitation given that such combination is not illogical or non-functional.

It should be understood that references to a single element are not necessarily so limited and may include one or more of such element. Any directional references (e.g., plus, minus, upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of embodiments.

Joinder references (e.g., attached, coupled, connected, and the like) are to be construed broadly and may include intermediate members between a connection of elements and relative movement between elements. As such, joinder references do not necessarily imply that two elements are directly connected/coupled and in fixed relation to each other. The use of “e.g.” throughout the specification is to be construed broadly and is used to provide non-limiting examples of embodiments of the disclosure, and the disclosure is not limited to such examples. Uses of “and” and “or” are to be construed broadly (e.g., to be treated as “and/or”). For example and without limitation, uses of “and” do not necessarily require all elements or features listed, and uses of “or” are intended to be inclusive unless such a construction would be illogical.

It is intended that all matter contained in the above description or shown in the accompanying drawings shall be interpreted as illustrative only and not limiting. Changes in detail or structure may be made without departing from the present disclosure.

Furthermore, the mixing and matching of features, elements and/or functions between various examples is expressly contemplated herein so that one of ordinary skill in the art would appreciate from this disclosure that features, elements, and/or functions of one example may be incorporated into another example as appropriate, unless described otherwise, above. Moreover, many modifications may be made to adapt a particular situation or material to the teachings of the present disclosure without departing from the scope thereof. Therefore, it is intended that the present teachings not be limited to the particular examples illustrated by the drawings and described in the specification, but that the scope of the present disclosure will include any embodiments falling within the foregoing description and the appended drawings.

What is claimed is:

1. A fluid sensor assembly, comprising:
a body including a fluid passage;
a first sensor connected to the body and directed toward the fluid passage; and
a second sensor connected to the body and directed toward the fluid passage;
wherein at least one of the first sensor and the second sensor is configured to transmit a signal into the fluid passage, and at least one of the first sensor and the second sensor is configured to receive at least a deflected version of the signal.

2. The fluid sensor assembly of claim 1, wherein the signal includes an ultrasonic signal.

3. The fluid sensor assembly of claim 2, wherein the ultrasonic signal includes a pulse.

4. The fluid sensor assembly of claim 1, wherein the first sensor includes a focused transmitting transducer and the second sensor includes a non-focused receiving transducer.

5. The fluid sensor assembly of claim 1, wherein the fluid passage includes a longitudinal axis and the first sensor is disposed at an oblique angle relative to the longitudinal axis.

6. The fluid sensor assembly of claim 5, wherein the oblique angle is between about 40 degrees and 50 degrees.

7. The fluid sensor assembly of claim 5, wherein the oblique angle is between about 44 and about 46 degrees.

8. The fluid sensor assembly of claim 1, comprising a coil disposed at least partially around the fluid passage, the coil including a longitudinal axis parallel to a longitudinal axis of the fluid passage.

9. The fluid sensor assembly of claim 1, wherein the first sensor is disposed at a first oblique angle relative to a longitudinal axis of the fluid passage and is disposed at a second oblique angle relative to a transverse axis of the body.

10. The fluid sensor assembly of claim 9, wherein the second sensor is aligned with the transverse axis of the body.

11. The fluid sensor assembly of claim 10, wherein the first sensor includes a focused transmitting transducer.

12. (canceled)

13. The fluid sensor assembly of claim 1, including a third sensor and a fourth sensor, wherein the third sensor is configured to transmit a second signal into the fluid passage and the fourth sensor is configured to receive at least one of a deflected version of the second signal and the deflected version of the signal.

14. The fluid sensor assembly of claim 1, comprising a third sensor configured to receive the deflected version of the signal.

15. The fluid sensor assembly of claim 14, wherein the third sensor is substantially coaxial with the second sensor.

16. The fluid sensor assembly of claim 1, comprising a fluid conduit connected to an inlet of the body, the fluid conduit including a vertical portion, a bent portion, and an outlet disposed at an oblique angle relative to a longitudinal axis of the fluid passage.

17. The fluid sensor assembly of claim 1, wherein the first sensor and the second sensor are disposed in a V-shaped configuration.

18. A method of sensing particles in a fluid, the method comprising:
providing a fluid sensor assembly, the fluid sensor assembly including:
a housing including a fluid passage;
a first sensor connected to the housing and directed toward the fluid passage; and
a second sensor connected to the housing and directed toward the fluid passage;
transmitting, via the first sensor, a signal into the fluid passage;
receiving, via the second sensor, a deflected version of the signal that has deflected or scattered after encountering a debris particle; and
detecting the debris particle according the deflected version of the signal;
wherein at least one of the first sensor and the second sensor is disposed at a first oblique angle relative to a longitudinal axis of the fluid passage and at a second oblique angle relative to a transverse axis of the housing.

19. (canceled)

20. The method of claim 18, comprising sensing the debris particle via an inductive sensor connected to the housing.

21. The method of claim 18, comprising:
providing an electronic control unit (ECU);
providing an inductive sensor; and
applying, via the ECU, signal processing methods to information from the first sensor, information from the second sensor, and information from the inductive sensor to determine a size of the debris particle and whether the debris particle includes metal.

22. The method of claim 18, comprising transmitting, via the second sensor, a second signal into the fluid passage; and receiving, via the first sensor, a deflected version of the second signal that has deflected or scattered after encountering a second debris particle.

23. The method of claim 18, comprising transmitting, via the first sensor, a second signal into the fluid passage; and receiving, via the second sensor, a deflected version of the second signal that has deflected or scattered after encountering the debris particle.
